(19) United States
(12) Patent Application Publication
(10) Pub. No.: US 2026/0283920 A1
(43) Pub. Date: Sep. 24, 2026

(54) DIBENZOYLMETHANE COMPOUNDS AS PHOTOCHEMICAL PRECURSORS OF ULTRAVIOLET ABSORBERS

(71) Applicant: ROKA FURADADA, S.L., BARCELONA (ES)

(72) Inventors: CRISTINA MOYA MUÑOZ, BARCELONA (ES); JOAN ATCHER UBIERGO, BARCELONA (ES); ÁNGEL MANUEL VALDIVIELSO PABLO, BARCELONA (ES); JUDIT CAMARGO SANROMÀ, BARCELONA (ES); JORDI HERNANDO CAMPOS, CORNELLÀ DE LLOBREGAT (ES); ROSA MARÍA SEBASTIÁN PÉREZ, RIPOLLET (ES)

(21) Appl. No.: 19/168,536

(22) PCT Filed: Mar. 27, 2024
(86) PCT No.: PCT/EP2024/058310

§ 371 Date: Sep. 24, 2025

(30) Foreign Application Priority Data
Mar. 28, 2023 (EP) 23382293.1

(51) Int. Cl. A61K 8/44; A61K 8/42; A61Q 17/04; C07C 233/88

(52) U.S. Cl. CPC A61K 8/445; A61K 8/42; A61Q 17/04; C07C 233/88

(57) ABSTRACT

Precursors for making dibenzoylmethane compounds, the precursors having an amido group in one of the rings and the corresponding photoconverted compounds. The compounds can be in the form of pharmaceutically or cosmetically acceptable salts, or stereoisomers of any of them or mixtures thereof, and can be used for the protection of either a human or animal living body or a material from ultraviolet radiation. Preparation processes for the compounds, as well as a cosmetic or pharmaceutical composition, or a personal care composition thereof.

This application claims the benefit of European Patent Application EP23382293 filed Mar. 28, 2023.

### TECHNICAL FIELD

This invention relates to photochemical precursors of ultraviolet absorbers, especially to some dibenzoylmethane compounds, as well as preparation process and uses thereof.

### BACKGROUND ART

Ultraviolet A (UVA, 320-400 nm) and ultraviolet B (UVB, 290-320 nm) invisible rays emitted from the sun can cause skin damage both immediate and long term. Some examples are sunburns, rashes, cell and tissue damage, and skin cancer. The increasing awareness of these risks has produced a change in sun tanning habits. This includes clothes, hats, eyeglasses, sunscreens, and other forms of photoprotection.

Sunscreen is any substance or material that protects the skin from the ultraviolet rays. Sunscreens are generally commercialized in the forms of spray, cream, topical lotion, ointment, and gel.

Several benzoic acid esters have been described as photochemical precursors of ultraviolet absorbers. Their photoprotective activity is due to the fact that are susceptible to be photochemically converted in situ to a sunscreen compound with UV protection ability.

The document Adaya Gallardo et al., “Dose-dependent progressive sunscreens. A new strategy for photoprotection?”, Photochemical & Photobiological Sciences, 2010, vol. 9, pp. 530-534 discloses that benzophenones absorb UV-B and UVA-II radiation while dibenzoylmethanes absorb UV-A thus preventing its penetration into the skin.

Benzophenones and dibenzoylmethanes have a prominent role in the UV filters currently approved for use in cosmetics. These families share an aromatic beta-hydrocabonyl in their structure which is a key component for their sun screening action.

Avobenzone, dioxybenzone, oxybenzone, sulisobenzone are some relevant broadly used sunscreen compounds.

When the radiation is absorbed, a phototautomerization is trigged forming an unstable enol which releases the absorbed radiation as heat. Each molecule participates in several cycles before being degraded.

Photostability may be defined as the response of a molecule to the exposure to solar, UV and visible light. This may vary depending on the solvent or the vehicle used.

Avobenzone is one of the most used filters. It provides protection through a large range of the UV-A spectrum, including UVA-1. Unfortunately, concerns regarding its photostability and the ability to degrade other sunscreen ingredients have been raised.

Avobenzone precursor, preavobenzone, is one of the most commonly used molecules. However, preavobenzone needs more than 20 minutes to fully reach complete photoconversion. It is therefore desirable to discover new sunscreen compounds that reach a complete photoconversion faster.

### SUMMARY OF INVENTION

Inventors have found that dybenzoylmethane compounds with an amido group in one of the rings show an enhanced photoconversion in comparison to the 1-(4-methoxyphenyl)vinyl 4-(tert-butyl)benzoate (preavobenzone).

An aspect of the present invention relates to a compound of formula (I), a pharmaceutically or a cosmetically acceptable salt thereof, or a stereoisomer of any of them or mixtures thereof,

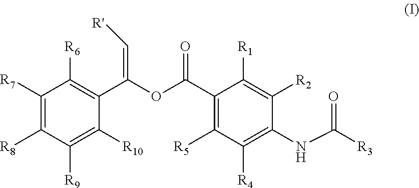

or, alternatively,

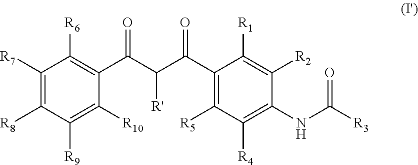

where: in compound (I), R′ is selected from the group consisting of H; (C₁-C₆)-alkyl, and (C₃-C₆)-cycloalkyl; R₁, R₂, R₄, R₅, R₆, R₇, R₉, and R₁₀ are a radical independently selected from the group consisting of H, hydroxy, amino, (C₁-C₆)-alkyl, (C₁-C₆)-alkoxy, (C₁-C₆)-alkylamino, and (C₁-C₆)-dialkylamino; R₃ is (C₁-C₆)-alkyl; and R₃ is selected form the group consisting of (C₁-C₆)-alkyl, (C₁-C₆)-alkoxy, hydroxy, amino, (C₁-C₆)-alkylamino, and (C₁-C₆)-dialkylamino; and in compound (I′), R₁ and R₆ are a radical independently selected from the group consisting of H, amino, (C₁-C₆)-alkyl, (C₁-C₆)-alkoxy, (C₁-C₆)-alkylamino, and (C₁-C₆)-dialkylamino; R′, R₂, R₄, R₅, R₇, R₉, R₁₀, R₃, and R₃ are as defined in compound (I).

Another aspect of the present invention relates to either a compound (I) or a compound (I′) as defined above, for use in the protection of either a human or animal living body, or a material, from ultraviolet radiation.

Another aspect of the present invention relates to a method for protecting a material from ultraviolet radiation which comprises treating the material with either a compound (I) or a compound (I′) as defined above.

Another aspect of the present invention relates to a composition selected from a cosmetic composition, a pharmaceutical composition, and a personal care composition comprising an effective amount for the protection of a human, or animal living body, from ultraviolet radiation, of a compound as defined above, together with one or more pharmaceutically or cosmetically acceptable excipients or carriers; or alternatively, a composition comprising at least an effective amount for the protection of a material from ultraviolet radiation, of a compound as defined above, together with appropriate vehicles.

Another aspect of the present invention relates to a process for preparing either a compound (I) or a compound (I′) as defined above, where: when compound is compound of formula (I), the process comprises a step of reacting a compound of formula (IIₐ), or an appropriate salt thereof, with a (C₁-C₆)-acyl halide, in the presence of an appropriate solvent and an appropriate base; wherein in compound (IIa), R′, R₁-R₁₀ are as defined in compound (I), and R₃ₐ′ is NH₂; and where when the compound is the compound of formula (I′), the process comprises irradiating the compound of formula (I) with UV light to yield compound of formula (I′),

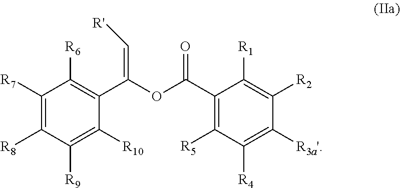

Finally, another aspect of the present invention relates to a compound of formula (II), a pharmaceutically or a cosmetically acceptable salt thereof, or a stereoisomer of any of them or mixtures thereof, where: in compound (II), R′ is selected from the group consisting of H; (C₁-C₆)-alkyl, and (C₃-C₆)-cycloalkyl; R₁, R₂, R₄, R₅, R₆, R₇, R₉, and R₁₀ are a radical independently selected from the group consisting of H, hydroxy, amino, (C₁-C₆)-alkyl, (C₁-C₆)-alkoxy, (C₁-C₆)-alkylamino, and (C₁-C₆)-dialkylamino; R₃′ is amino or nitro; and R₃ is selected form the group consisting of (C₁-C₆)-alkyl, (C₁-C₆)-alkoxy, hydroxy, amino, (C₁-C₆)-alkylamino, and (C₁-C₆)-dialkylamino,

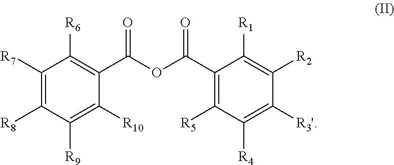

### BRIEF DESCRIPTION OF DRAWINGS

FIG. 1 a) and b) are UV-Vis spectra which show the absorption changes of a Preavobenzone (PvB 360) (a) and 1-(4-methoxyphenyl)vinyl 4-acetamidobenzoate (PVB-1) (b) when a 3×10⁻⁵ M solution in MeOH is irradiated at 312 nm.

FIG. 2 shows the SPF and UVA-PF of PvB 360 (Preavobenzone, comparative compound) and PVB-1 (compound of the present invention) at different irradiation times. (PVB-1) corresponds to compound (I) where, R′ is H, R₁, R₂, R₄, R₅, R₆, R₇, R₉, and R₁₀ are H; R₃ is methyl; and R₃ is methoxyl.

### DETAILED DESCRIPTION OF THE INVENTION

For the purposes of the invention, any ranges given include both the lower and the upper endpoints of the range. Ranges given, such as temperatures, times, sizes, and the like, should be considered approximate, unless specifically stated.

The term “room temperature” as disclosed herein refers to a temperature of the environment, without heating or cooling, and is generally comprised of from 20 to 25° C.

The terms “(C₁-C₆)-alkyl”, “(C₁-C₆)-alkoxy”, “(C₁-C₆)-alkylamino”, and “(C₁-C₆)-dialkylamino” shall be construed as straight or branched chains.

The term “pharmaceutically or cosmetically acceptable salts” used herein encompasses any salt formed from pharmaceutically or cosmetically acceptable non-toxic acids including inorganic or organic acids. There is no limitation regarding the salts, except that if used for therapeutic purposes, they must be pharmaceutically or cosmetically acceptable.

As some of the compounds of formula (I) are basic compounds, salts may be prepared from pharmaceutically acceptable non-toxic acids, including inorganic and organic acids. Such acids include for instance acetic, benzenesulfonic, benzoic, camphorsulfonic, citric, ethansulfonic, fumaric, gluconic, glutamic, hydrobromic, hydrochloric, lactic, maleic, malic, malonic, mandelic, methanesulfonic, phosphoric, succinic, sulfuric, nitric, adipic, aspartic, 1,5-naphtalendisulfonic, oxalic, pivalic, propionic, tartaric, p-toluensulfonic acid, and the like.

The preparation of pharmaceutically acceptable salts of the compounds of formula (I), of formula (I′), or of formula (II), including compounds of formula (IIa) or (IIb), can be carried out by methods known in the art. For instance, they can be prepared from the parent compound, which contains a basic moiety, by conventional chemical methods. Generally, such salts are, for example, prepared by reacting the base form of these compounds with a stoichiometric amount of the appropriate pharmaceutically acceptable acid in water or in an organic solvent or in a mixture of them.

The compounds of the invention may be in crystalline form either as free solvation compounds or as solvates (e.g., hydrates) and it is intended that both forms are within the scope of the present invention. Methods of solvation are generally known within the art.

The term “solvate” refers to a molecular complex comprising any of the compounds of formula of formula (I), of formula (I′), or of formula (II), including compounds of formula (IIₐ) or (II_{b}) or any of their salts, and a stoichiometric or non-stoichiometric amount of one or more solvent molecules bound by non-covalent intermolecular forces. When the one or more solvent molecules forming part of the molecular complex is water, the solvate is a hydrate.

The expression “effective amount for the protection of a human or animal living body, or a material, from ultraviolet radiation” as used herein, refers to the amount of a compound that, when administered, is sufficient to protect from UV radiation. In the case of human or animal living body this means that prevent skin damage. Thus, the effective amount as used herein refers to a therapeutically effective amount.

The expression “pharmaceutically or cosmetically acceptable excipients or carriers” refers to pharmaceutically acceptable materials, compositions, or vehicles. Each component must be pharmaceutically or cosmetically acceptable in the sense of being compatible with the other ingredients of the pharmaceutical or cosmetical composition. It must also be suitable for use in contact with the skin of humans and animals without excessive toxicity, irritation, allergic response, immunogenicity or other problems or complications commensurate with a reasonable benefit/risk ratio.

The term “cosmetically acceptable” which is herein used interchangeably refers to that excipients or carriers suitable for use in contact with skin or human and animals without undue toxicity, incompatibility, instability, allergic response, among others.

As mentioned above, one aspect of the present invention relates to a compound of formula (I) or of formula (I′), their pharmaceutically or cosmetically acceptable salts, or their stereoisomers of any of them or mixtures thereof) as defined above.

In a particular embodiment, the compound of the present invention is the compound of formula (I), or a pharmaceutically or a cosmetically acceptable salt thereof, or a stereoisomer of any of them or mixtures thereof.

In a particular embodiment, compounds of formula (I) are those where R₁ and R₆ are a radical independently selected from the group consisting of H, amino, (C₁-C₆)-alkyl, (C₁-C₆)-alkoxy, (C₁-C₆)-alkylamino, and (C₁-C₆)-dialkylamino.

In another particular embodiment, the compounds of the present invention of formula (I) or alternatively, of formula (I′) are those where R₁, R₂, R₄, R₅, R₆, R₇, R₉, and R₁₀ are H and R₈ is (C₁-C₆)-alkoxyl, and R′ is H. In another particular embodiment, the compounds are those where R₃ is (C₁-C₃)-alkyl. In another particular embodiment, the compounds of the present invention are those where in formula (I) and in formula (I′), R′, R₁, R₂, R₄, R₅, Re, R₇, R₉, and R₁₀ are H; R₃ is methyl; and R₃ is methoxyl.

It is also part of the invention a compound as defined above, for use in the protection of either a human or animal living body, from ultraviolet radiation. This aspect of the present invention may also be formulated as the use of compound (I) or (I′) as defined above for the manufacture of a cosmetic composition, a pharmaceutical composition, or a personal care composition for the protection of a human, or animal living body, from ultraviolet radiation. The present invention also relates to a method for the protection of a human, or animal living body, from ultraviolet radiation, comprising administering an effective amount for the protection of a human, or animal living body, from ultraviolet radiation, of a compound as defined above, together with one or more pharmaceutically or cosmetically acceptable excipients or carriers. In a particular embodiment, the animal living body is a mammal.

The use of a compound as defined above for the protection of a human, or animal living body, from ultraviolet radiation is considered part of the invention. In particular, the use as defined above, wherein it comprises photochemically converting compound of formula (I) in situ to a sunscreen compound.

It is also part of the invention a method for protecting a material from ultraviolet radiation which comprises treating the material with either a compound (I) or a compound (I′) as defined above. Generally, the method for protecting the material comprises applying an effective amount of either the compound (I) or the compound (I′) as defined above for protecting a material from ultraviolet radiation.

The material may be selected from the group consisting of organic compounds, oils, fats, waxes, gelatins, sunscreens, polymers, such as polyolefins, polyketones, polystyrene, polyvinyl chloride (PVC), polyacrylates, polymethacrylates, polyacrylamides, polyacrylonitriles, polyvinyl alcohol derivatives, polyvinyl acetate derivatives, polyurethanes, polyamides, polyesters, polyureas, polycarbonates, polysiloxanes, polyketimines, radiation curable compositions, resins, such as hydrocarbon resins, phenol/formaldehyde resins, urea/formaldehyde resins, melamine/formaldehyde resins, unsaturated polyester resins, crosslinkable acrylic resins, crosslinked epoxy resins, epoxy/melamine resins, varnishes, cellulose, cellulose-based paper formulations, photographic materials, photographic film paper, metallic products, ceramic products, biocides, fibers, textile fabrics, dyes, inks, coatings, adhesives, leathers, woods, lenses, composites, mixtures or blendslike.

The term “effective amount of either the compound (I) or the compound (I′) for protecting a material from ultraviolet radiation” as used herein refers to the amount of a compound that, when applied to a material, is sufficient to protect from UV radiation, i.e., an effective amount which prevent or minimizes the degradation of the material.

In a particular embodiment of the method, either the compound (I) or the compound (I′) as defined above is applied in the form of a composition which further comprises appropriate components selected from at least a polymer, a solvent, an additive, and a mixture thereof. Examples of additives are antioxidants, preservatives, thickeners, colouring agents, or opacifying agents. In a particular embodiment of the method, the composition used in the method is an industrial composition.

In another particular embodiment, the compound of the present invention is compound of formula (I) as defined above, where said compound of formula (I) is susceptible to be photochemically converted in situ to a sunscreen compound.

In another particular embodiment, the compound of the present invention of formula (I) or formula (I′) for use as defined above, are those where the protection is for the UV-A and UV-B.

In another particular embodiment, the compound for use as defined above, are those where the protection is for the UV radiation emitted by the sun.

In another particular embodiment, the compound for use as defined above, are those which are characterized by a progressive UV protection depending on the time to sun exposition and the degree of sun radiation.

The compounds of formula (I) or of formula (I′) of the present invention may be in the form of a composition selected from a cosmetic composition, a pharmaceutical composition, and a personal care composition comprising an effective amount for the protection of a human, or animal living body, or a material, from ultraviolet radiation, of such compounds, together with one or more pharmaceutically or cosmetically acceptable excipients or carriers.

The particular embodiments of the first aspect of the invention mentioned above are also considered particular embodiments of the composition of the present invention.

In a particular embodiment, the cosmetic composition, the pharmaceutical composition, or the personal care composition as defined above are those where the effective amount of the compound of formula (I) or (I′) ranges from 0.1 to 10 wt. % based on the total weight of the composition. In another particular embodiment, the cosmetic composition, the pharmaceutical composition, or the personal care composition as defined above, are those where the effective amount of the compound of formula (I) or (I′) ranges from 0.5 to 8 wt. % based on the total weight of the composition. In another particular embodiment, the cosmetic composition, the pharmaceutical composition, or the personal care composition are those where the effective amount of the compound of formula (I) or (I′) ranges from 1 to 6 wt. % based on the total weight of the composition.

In another particular embodiment, the compositions of the present invention are those comprising an effective amount of a dibenzoylmethane compound of formula (I), a pharmaceutically or a cosmetically acceptable salt thereof, or a stereoisomer of any of them or mixtures thereof, where the composition has enhanced photoconversion.

In another particular embodiment, the compounds of the present invention may also be employed to form a photostable composition. Such photostable compositions may further include a variety of other components known in the art including triazines, benzotriazoles, hindered amine light stabilizers, radical scavengers, antioxidants and the like.

The composition of the present invention is for topical administration. In a particular embodiment, the cosmetic composition, the pharmaceutical composition, or the personal care composition as defined above, are those where the composition is selected from the group consisting of creams, ointments, oils, lotions, gels, sticks, foams, milks, suspensions, powders, emulsions, dispersions, sprays, aerosols, lipsticks, foundations, makeup, loose or press powders, eye blushes, eye shadows, mascaras, nail varnishes, nail lacquers and non-permanent dyeing composition for the hair.

In another particular embodiment, the compositions of the present invention can contain one or more additional organic sunscreen agents for filtering UV-A or UV-B.

In another particular embodiment, the composition is a sunscreen composition. In another particular embodiment, the sunscreen composition may have a sun protection factor (SPF) equal to or higher than 6, preferably, equal to or higher than 30, more preferably, equal to or higher than 50. Preferably, the sunscreen composition comprises physical, chemical filters, or combinations thereof.

Appropriate excipients or carriers for the composition of the present invention are for instance, dispersing agents, preservatives, anti-foams, perfumes, fragrances, oils, waxes, propellants, dyes, pigments, emulsifiers, surfactants, thickeners, humectants, exfoliants and emollients.

The topical compositions of the present invention can be produced by conventional processes known in the art, such as for instance by mixing the different components of the composition, in any order. The appropriate excipients and/or carriers, and their amounts, can readily be determined by those skilled in the art according to the type of the composition being prepared.

It is part of the invention, a composition comprising at least an effective amount for the protection of a material from ultraviolet radiation, of a compound as defined above, together with appropriate components selected from at least a polymer, a solvent, an additive, and a mixture thereof. Examples of additives are antioxidants, preservatives, thickeners, colouring agents, or opacifying agents. The composition may be an industrial composition which comprises the compound as defined above.

In a particular embodiment, the composition for the protection of a material from ultraviolet radiation as defined above is that where the effective amount of the compound of formula (I) or (I′) ranges from 0.1 to 10 wt. % based on the total weight of the composition. In another particular embodiment, the composition as defined above is that where the effective amount of the compound of formula (I) or (I′) ranges from 0.5 to 8 wt. % based on the total weight of the composition. In another particular embodiment, the composition is that where the effective amount of the compound of formula (I) or (I′) ranges from 1 to 6 wt. % based on the total weight of the composition.

The compounds of formula (I) of the present invention can be prepared by a process comprising a step of reacting a compound of formula (IIₐ) or an appropriate salt thereof; with a (C₁-C₆) acyl halide such as acetyl chloride or a (C₂-C₁₂) acid anhydride such as acetic anhydride in the presence of an appropriate solvent and an appropriate base; wherein in compound of formula (IIₐ), R′, R₁-R₁₀ are as defined in compound (I) and R₃ₐ′ is NH₂;

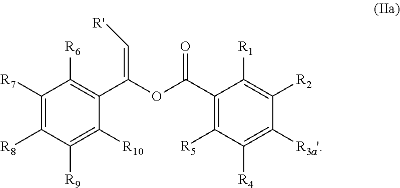

In a particular embodiment of the process, the appropriate solvent for the previous step is selected from the group consisting of (C₆-C₈)-aromatic hydrocarbons such as toluene or xylene, or (C₁-C₃)-chlorine containing solvents such as dichloromethane, or dichloroethane. In another particular embodiment of the process, the appropriate base is a tertiary amine such as triethylamine alone or with 4-dimethylaminopyridine, or N,N-diisopropylethylamine. This step of the process is generally carried out at a temperature comprised in a range from −20° C. to 0° C.

In another particular embodiment, the process as defined above, further comprises a previous step which comprises submitting a compound of formula (II_{b}) to a reduction reaction to yield a compound of formula (IIₐ) as defined above, wherein in compound of formula (II_{b}), R′, R₁-R₁₀ are as defined in compound (I) and R_{3b}′ is NO₂.

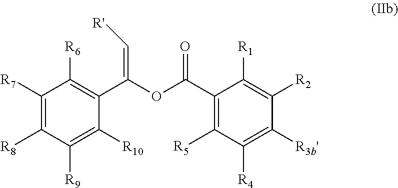

The reduction reaction may be carried out with a reducing agent such as an oxidized metal in the presence of an acid. Examples of appropriate metals are for instance, Fe, Sn or Zn. Examples of appropriate acids are for instances chlorohydric acid or acetic acid. In a particular embodiment, the metal is zinc. In another particular embodiment of the process, this step is carried out in the presence of an appropriate solvent which can be a (C₁-C₃) chlorine containing solvent such as chloroform or dichloromethane. In another particular embodiment the preferred ratio of (C₁-C₃) chlorine containing solvent and acid is 11:1,5 or 7:0,5 respectively. This step of the process is generally carried out at a temperature comprised in a range from −20° C. to 0° C.

In another particular embodiment, the process of the present invention as defined above, further comprises a previous step which comprises submitting a compound of formula (III) to an elimination reaction to yield a compound of formula (II_{b}), wherein in compound (Ill) X is a halide, R₁-R₁₀ are as defined in compound (I) and R_{3b}′ is NO₂,

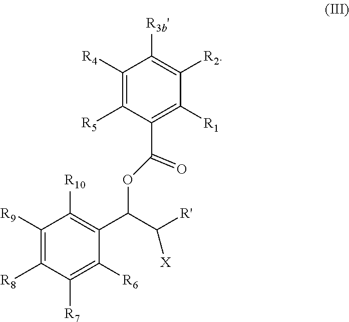

In a particular embodiment, X is a halogen selected from the group consisting of Br, Cl, and I. In a particular embodiment of the process, the compound of formula (III) is that where X is bromine.

The compound of formula (III) can be prepared from 2-halo-1-(4-methoxyphenyl)ethan-1-one. The term halo means any of Cl, Br, and I. The compound may be submitted to a reduction reaction with a reducing agent such as NaBH₄ in the presence of an appropriate solvent. Generally, the reaction is carried out at low temperatures such as (−20° C.)−10° C. The solvent may be an (C₁-C₄)-alcohol such as methanol to yield 2-halo-1-(4-methoxyphenyl)ethan-1-ol. The compound thus obtained can be reacted with a p-nitrobenzoyl halide such as p-nitrobenzoyl chloride in the presence of an appropriate solvent and an appropriate base. An example of an appropriate solvent is tetrahydrofuran. Examples of appropriate bases are tertiary amines such as triethylamine, alone or with 4-dimethylaminopyridine.

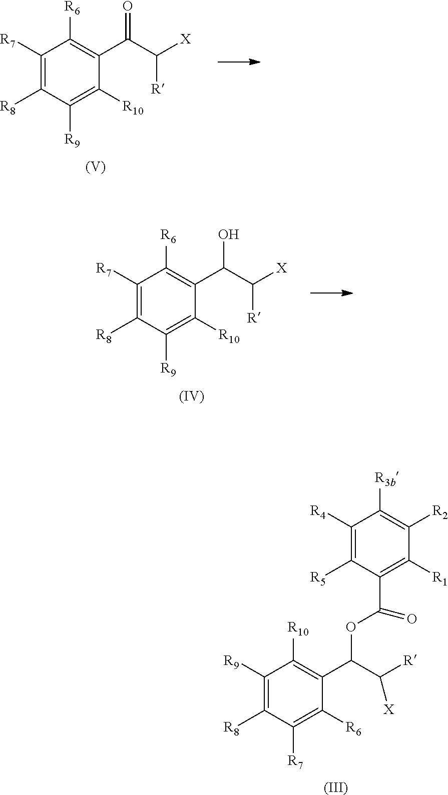

In the compounds of formula (III), formula (IV), and formula (V), R₁-R₁₀ are as defined in compound (I), X is a halogen selected from the group consisting of Br, Cl, and I, and R_{3b}′ is NO₂.

The compounds of formula (I′) of the present invention can be prepared by a process comprising irradiating the compound of formula (I) with UV light to yield the compound of formula (I′) of the present invention.

Compounds of formula (IIₐ) and (II_{b}) are also part of the invention. They can also be in the form of a pharmaceutically or a cosmetically acceptable salt thereof, or a stereoisomer or any of them or mixtures thereof. The salts of compound of formula (IIₐ) or (II_{b}) refers to non-toxic salts. As some of the compounds of formula (IIₐ) or (II_{b}) are basic compounds, salts may be prepared with pharmaceutically or cosmetically acceptable non-toxic acids.

Throughout the description and claims the word “comprise” and variations of the word, are not intended to exclude other technical features, additives, components, or steps. Furthermore, the word “comprise” encompasses the case of “consisting of”. Additional objects, advantages and features of the invention will become apparent to those skilled in the art upon examination of the description or may be learned by practice of the invention. The following examples and drawings are provided by way of illustration, and they are not intended to be limiting of the present invention. Furthermore, the present invention covers all possible combinations of particular and preferred embodiments described herein.

### EXAMPLES

### Example 1: Preparation of 2-bromo-1-(4-methoxyphenyl)ethan-1-ol

In a 100 mL round-bottom flask, 3 g of BMAP (13.2 mmol, 1 eq.) were dissolved in 60 mL of MeOH. Then, 1 g of NaBH₄ (26.4 mmol, 8 eq.) was added portion-wise at −10° C. (ice-salt bath). The solution was stirred at this temperature for 1 h (until completion by TLC). Afterwards, 100 mL of DCM and 150 mL of 1 M aqueous HCl were added. The resulting mixture was stirred at 0° C. for 30 min. The organic phase was decanted, and the aqueous phase was extracted twice with DCM. The organic phases were combined, washed with brine, and dried over anhydrous Na₂SO₄. The solvent was evaporated under reduced pressure to yield the product as a colorless oil (2.86 g, 94% yield).

### Example 2: Preparation of 2-bromo-1-(4-methoxyphenyl)ethyl 4-nitrobenzoate

2-bromo-1-(4-methoxyphenyl)ethan-1-ol (2.5 g, 10.82 mmol, 1 eq.) was dissolved in 50 mL of dry THF under inert atmosphere of N₂. Then, the mixture was cooled down to 0° C. (ice bath) and 2.26 mL of NEt₃ (16.23 mmol, 1.5 eq.) were added. Finally, a solution of 4 g of p-NBCI (21.64 mmol, 2.0 eq.) in 7 mL of dry THF was added dropwise using an addition funnel. After stirring at 0° C. for 10 min, the mixture was stirred at room temperature for 7 h, after which complete conversion of the starting material was observed by ¹H-NMR. Then, volatiles were partially evaporated under reduced pressure and the crude mixture was washed with saturated aqueous NaHCO₃. The resulting organic phase was dried over anhydrous Na₂SO₄, and the solvent evaporated under reduced pressure. The product was purified with silica gel column chromatography using Hexanes/DCM (from 7:3 to 6:4) as the mobile phase to yield the product as a yellowish oil (3.709 g, 90% yield). ¹H-NMR (300 MHz; Chloroform-d) δ ppm 8.35-8.21 (m, 4H), 7.42-7.33 (m, 2H), 6.98-6.88 (m, 2H), 6.18 (dd, J₁=8.5 Hz, J₂=4.4 Hz, 1H), 3.83 (dd, J₁=10.9 Hz, J₂=8.5 Hz, 1H) 3.71 (dd, J₁=10.9 Hz, J₂=4.4 Hz, 1H). ¹³C-NMR (75 MHz; Chloroform-d) δ ppm 163.53 (1C), 160.08 (1C), 150.54 (1C), 135.12 (1C), 130.80 (2C), 129.06 (1C), 127.96 (2C), 123.51 (2C), 114.17 (2C), 76.16 (1C), 55.20 (1C), 34.14 (1C).

### Example 3: Preparation of 1-(4-methoxyphenyl)vinyl 4-nitrobenzoate

2-bromo-1-(4-methoxyphenyl)ethyl 4-nitrobenzoate (2.14 g, 5.64 mmol, 1.0 eq.) was introduced in a three-neck round-bottom flask and dissolved in 50 mL of anhydrous toluene. Then, the mixture was heated to reflux and 1.7 mL of DBU (11.28 mmol, 2.0 eq.) were added slowly. The mixture was stirred at reflux temperature for 1 h. Afterwards, the mixture was cooled down to room temperature and the reaction was quenched with 60 mL of 0.5 M aqueous HCl. The phases were separated, and the aqueous phase was extracted with ethyl acetate. The organic phases were combined, washed with brine, and dried over anhydrous Na₂SO₄. The product was obtained as an orange-colored solid (1.501 g, 90% yield) after evaporation of the solvent under reduced pressure. ¹H-NMR (300 MHz; Chloroform-d) δ ppm 8.36 (s, 4H), 7.48-7.39 (m, 2H), 6.93-6.84 (m, 2H), 5.49 (d, J=2.5 Hz, 1H), 5.09 (d, J=2.5 Hz, 1H), 3.81 (s, 3H). ¹³C-NMR (75 MHz; Chloroform-d) δ ppm 163.02 (1C), 160.43 (1C),152.83 (1C), 150.90 (1C), 134.92 (1C), 131.25 (2C), 126.36 (2C), 126.33 (1C), 123.79 (2C), 114.09 (2C), 100.83 (1C), 55.35 (1C).

### Example 4: Preparation of 1-(4-methoxyphenyl)vinyl 4-aminobenzoate

1-(4-methoxyphenyl)vinyl 4-nitrobenzoate (507.6 mg, 1.65 mmol, 1.0 eq.) was dissolved in 50 mL of CHCl₃/Acetic acid 9:1 in a 100 mL round-bottom flask. The mixture was cooled down to <0° C. (ice-salt bath) and then, 2.15 g of zinc (33 mmol, 20 eq.) were added portion-wise. The solution was stirred at <0° C. and after 20 min TLC analysis (Hexanes/DCM, 4:6) showed that no starting material remained in the reacting mixture. Afterwards, the excess of zinc was removed by filtration with Celite®. Saturated aqueous NaHCO₃ (50 mL) was added to the resulting filtrate and the mixture was stirred for 15 min. Then, the organic layer was separated and washed with more saturated aqueous NaHCO₃. The aqueous phases were extracted with CHCl₃. The organic phases were combined, dried over anhydrous Na₂SO₄ and the solvent evaporated under reduced pressure to yield the product as a brown-yellowish solid (410 mg, 92% yield). ¹H-NMR (300 MHz; Chloroform-d) δ ppm 8.05-7.94 (m, 2H), 7.51-7.41 (m, 2H), 6.91-6.79 (m, 2H), 6.74-6.63 (m, 2H), 5.43 (d, J=2.0 Hz, 1H), 5.01 (d, J=2.0 Hz, 1H), 4.14 (broad s, 2H), 3.80 (s, 3H). ¹³C-NMR (75 MHz; Chloroform-d) δ ppm 165.05 (1C), 160.19 (1C), 153.17 (1C), 151.58 (1C), 132.42 (2C), 127.45 (1C), 126.47 (2C), 118.85 (1C), 114.00 (4C), 100.29 (1C), 55.42 (1C).

### Example 5: Preparation of 1-(4-methoxyphenyl)vinyl 4-acetamidobenzoate

In a 100 mL Schlenk, 303.0 mg (1.13 mmol, 1.0 eq.) of 1-(4-methoxyphenyl)vinyl 4-aminobenzoate were dissolved in 30 mL of dry DCM under inert atmosphere of N₂. Then, 0.24 mL NEt₃ (1.688 mmol, 1.5 eq.) were added. Afterwards, 0.104 mL of CH₃COCl (1.46 mmol, 1.3 eq.) were added dropwise into the mixture at <0° C. (ice-salt bath). The mixture was stirred at <0° C. for 1 h, after which complete conversion of the starring material was observed by TLC. Then, 30 mL of saturated aqueous NH₄Cl were added to quench the excess of acid chloride. The organic layer was separated and washed with more saturated aqueous NH₄Cl. The aqueous phase was extracted with DCM and the organic layers were combined, washed with brine, dried over anhydrous Na₂SO₄ and the solvent evaporated under reduced pressure. ¹H-NMR (300 MHz; Chloroform-d) δ ppm 8.20-8.15 (m, 2H), 7.73-7.63 (m, 2H), 7.53-7.42 (m, 1H), 7.39 (s, 1H), 6.94-6.83 (m, 2H), 5.48 (d, J=2.2 Hz, 1H), 5.06 (d, J=2.2 Hz, 1H), 3.82 (s, 3H), 2.25 (s, 3H). ¹³C-NMR (75 MHz; Chloroform-d) δ ppm 169.01 (1C), 164.74 (1C), 160.33 (1C), 153.08 (1C), 143.06 (1C), 131.49 (2C), 126.94 (2C), 126.41 (2C), 124.68 (1C), 119.09 (1C), 114.08 (2C), 100.51 (1C), 55.42 (1C), 24.80 (1C).

### Example 6: Photoactivation of 1-(4-methoxyphenyl)vinyl 4-acetamidobenzoate

1-(4-methoxyphenyl)vinyl 4-acetamidobenzoate (102.9 mg, 0.33 mmol) was dissolved in 50 mL of degassed MeOH in a photochemical reactor. Then, the solution was irradiated with a high-pressure mercury lamp (HPK125W) for 2 hours (refrigeration temperature=−15° C.) under inert atmosphere. Afterwards, the solvent was evaporated and the product purified by means of a silica gel column chromatography using hexanes/ethyl acetate (7:3) as the mobile phase. Photoconverted product was obtained as slightly yellow solid (42.6 mg, 42% yield).

### Example 7: Comparison of the photochemical properties of 1-(4-methoxyphenyl)vinyl 4-acetamidobenzoate and preavobenzone

The UV-Vis spectra of (a) preabovenzone, PvB-360, (comparative example) and (b) 1-(4-methoxyphenyl)vinyl 4-acetamidobenzoate, PVB-1, (compound according to the invention) when a 3×10⁻⁵ M solution in MeOH is irradiated at 312 nm was obtained. This UV-Vis spectra show the absorption changes of both compounds,

The main difference is that 1-(4-methoxyphenyl)vinyl 4-acetamidobenzoate is photoconverted into the subsequent photoproduct faster than avobenzone. After 10 min of irradiation, 1-(4-methoxyphenyl)vinyl 4-acetamidobenzoate is fully converted into the photoproduct but avobenzone needs more than 20 min to reach complete photoconversion.

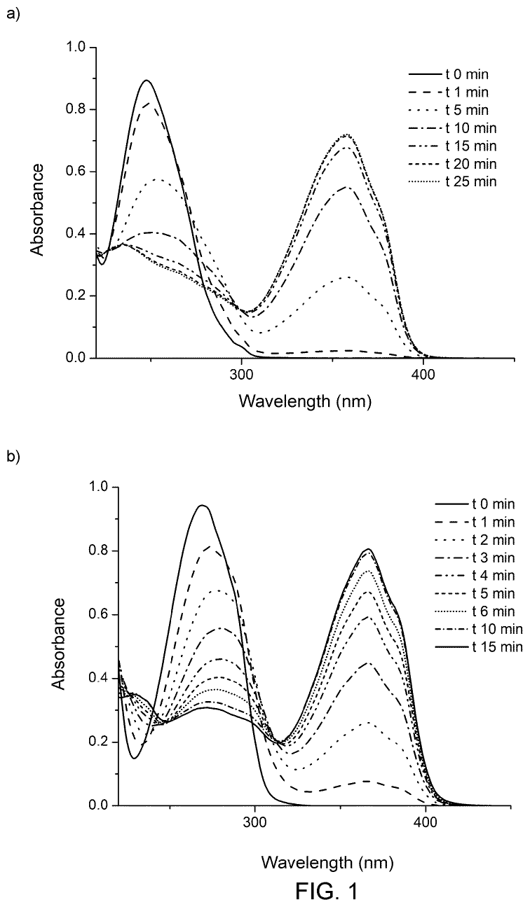

Moreover, 1-(4-methoxyphenyl)vinyl 4-acetamidobenzoate has the maximum of absorption at 270 nm, and it can absorb more UV-B radiation than avobenzone (see FIG. 1 (a) and (b))

### Example 8: Activation Study by Irradiation Using a Solar Simulator of PvB-360 (Comparative Example) and PVB-1 (Example of the Invention)

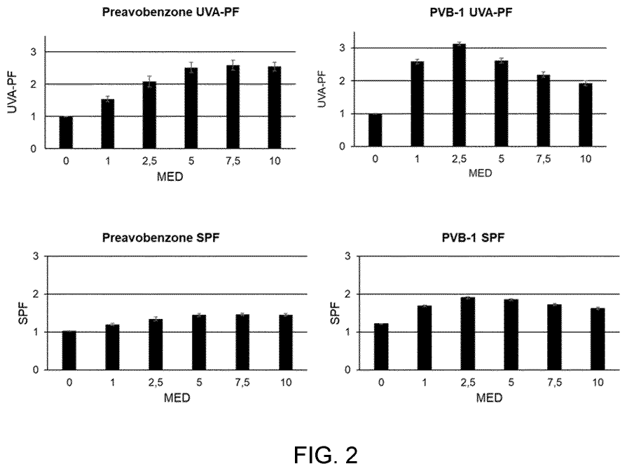

An activation study was performed by irradiate, using a solar simulator, a thin layer of the filter in the top of a PMMA plate dissolved in a proper solvent. In this case, dibutyl adipate as a solvent was used (filter 2% w/w). It was possible to measure the SPF and UVA-PF of (Preavobenzone) and PVB-1 at different irradiation times (measured in MEDs) by means of a Labsphere® instrument (see FIG. 2 SPF and UVA-PF of PvB-360 (Preavobenzone) and PVB-1 at different irradiation times).

It was observed that the SPF values are higher for PVB-1 in comparison with PvB360. Regarding UVA-PF values, it was observed that PVB-1 reaches higher values at the same dose of irradiation. In fact, PVB-1 reached just over 3 UVA-PF units when irradiated for 2.5 MEDs. Meanwhile, the maximum value for PvB-360 was achieved after irradiating 5 MEDs and its value was around 2.5 units.

### CITATION LIST

### Patent Literature

WO2006100225

### Non Patent Literature

Adaya Gallardo et al., “Dose-dependent progressive sunscreens. A new strategy for photoprotection?”, Photochemical & Photobiological Sciences, 2010, vol. 9, pp. 530-534

What is claimed is:

1. A compound of formula (I), a pharmaceutically or a cosmetically acceptable salt thereof, or a stereoisomer of any of them or mixtures thereof, 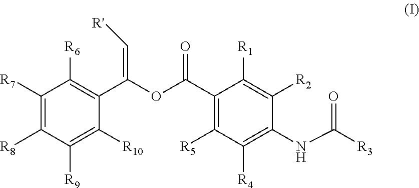
or, alternatively, 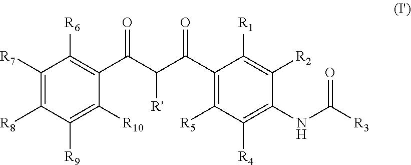
wherein:
in compound (I),
R′ is selected from the group consisting of H; (C₁-C₆)-alkyl, and (C₃-C₆)-cycloalkyl;
R₁, R₂, R₄, R₅, R₆, R₇, R₉, and R₁₀ are a radical independently selected from the group consisting of H, hydroxy, amino, (C₁-C₆)-alkyl, (C₁-C₆)-alkoxy, (C₁-C₆)-alkylamino, and (C₁-C₆)-dialkylamino;
R₃ is (C₁-C₆)-alkyl; and
R₈ is selected form the group consisting of (C₁-C₆)-alkyl, (C₁-C₆)-alkoxy, hydroxy, amino, (C₁-C₆)-alkylamino, and (C₁-C₆)-dialkylamino; and
in compound (I′),
R₁ and R₆ are a radical independently selected from the group consisting of H, amino, (C₁-C₆)-alkyl, (C₁-C₆)-alkoxy, C₁-C₆)-alkylamino, and (C₁-C₆)-dialkylamino;
R′, R₂, R₄, R₅, R₇, R₉, R₁₀ R₃ and R₃ are as defined in compound (I).

2. The compound according to claim 1, which is the compound of formula (I), or a pharmaceutically or a cosmetically acceptable salt thereof, or a stereoisomer of any of them or mixtures thereof.

3. The compound according to claim 1, wherein R₁, R₂, R₄, R₅, R₆, R₇, R₉, and R₁₀ are H and R₈ is (C₁-C₆)-alkoxyl, and R′ is H.

4. The compound according to claim 1, wherein R₃ is (C₁-C₃)-alkyl.

5. The compound according to claim 1, wherein in formula (I) and formula (I′) R′, R₁, R₂, R₄, R₅, R₆, R₇, R₉, and R₁₀ are H; R₃ is methyl; and R₃ is methoxyl.

6. A method for the protection of a human or animal living body from ultraviolet radiation comprising administering an effective amount of a compound as defined in claim 1, together with one or more pharmaceutically or cosmetically acceptable excipients or carriers.

7. A method for protecting a material from ultraviolet radiation which comprises treating the material by a composition comprising an effective amount of a compound as defined in claim 1.

8. A method according to claim 6, wherein the method comprises photochemically converting the compound of formula (I) or formula (I′) in situ to a sunscreen compound.

9. A composition comprising the compound as defined in claim 1, wherein the composition is either
(i) a composition selected from the group consisting of a cosmetic composition, a pharmaceutical composition, and a personal care composition and comprises at least an effective amount of the compound as defined in claim 1 for the protection of either a human or animal living body from ultraviolet radiation, together with one or more pharmaceutically or cosmetically acceptable excipients or carriers; or alternatively,
a composition comprising at least an effective amount of the compound as defined in claim 1 for the protection of a material from ultraviolet radiation, together with appropriate components selected from at least a polymer, a solvent an additive, and a mixture thereof.

10. The cosmetic or pharmaceutical composition or a personal care composition according to claim 9, wherein the effective amount ranges from 0.1 to 10 wt. % based on the total weight of the composition.

11. The composition for the protection of a material from ultraviolet radiation according to claim 9, wherein the effective amount ranges from 0.1 to 10 wt. % based on the total weight of the composition.

12. The cosmetic or pharmaceutical composition or a personal care composition according to claim 9, wherein the composition is selected from the group consisting of creams, ointments, oils, lotions, gels, sticks, foams, milks, suspensions, powders, emulsions, dispersions, sprays, aerosols, lipsticks, foundations, makeup, loose or press powders, eye blushes, eye shadows, mascaras, nail varnishes, nail lacquers and non-permanent dyeing composition for the hair.

13. A process for preparing a compound as defined in claim 1,
wherein:
when compound is compound of formula (I), the process comprises a step of reacting a compound of formula (IIₐ) or an appropriate salt thereof; 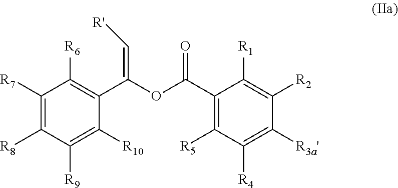
with a (C₁-C₆) acyl halide in the presence of an appropriate solvent and an appropriate base;
wherein R′, R₁-R₁₀ are as defined in compound (I) and R₃ₐ′ is NH₂; and
wherein when the compound is the compound of formula (I′), the process comprises irradiating the compound of formula (I) with UV light to yield compound of formula (I′).

14. The process according to claim 13, further comprising a previous step which comprises submitting a compound of formula (II_{b}) to a reduction reaction to yield a compound of formula (IIₐ), 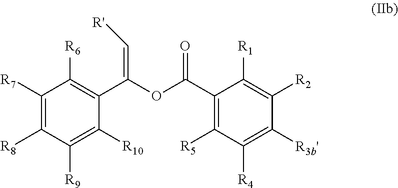
wherein in compound of formula (II_{b}), R′, R₁-R₁₀ are as defined in compound (I) and R_{3b}′ is NO₂.

15. A compound of formula (II), a pharmaceutically or a cosmetically acceptable salt thereof, or a stereoisomer of any of them or mixtures thereof, 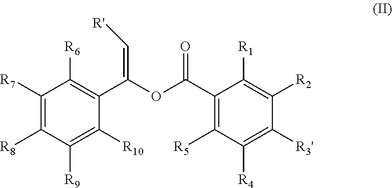
wherein:
in compound (II),
R′ is selected from the group consisting of H; (C₁-C₆)-alkyl, and (C₃-C₆)-cycloalkyl;
R₁, R₂, R₄, R₅, R₆, R₇, R₉, and R₁₀ are a radical independently selected from the group consisting of H, hydroxy, amino, (C₁-C₆)-alkyl, (C₁-C₆)-alkoxy, (C₁-C₆)-alkylamino, and (C₁-C₆)-dialkylamino;
R₃′ is amino or nitro; and
R₈ is selected form the group consisting of (C₁-C₆)-alkyl, (C₁-C₆)-alkoxy, hydroxy, amino, (C₁-C₆)-alkylamino, and (C₁-C₆)-dialkylamino.

16. The compound according to claim 2, wherein R₁, R₂, R₄, R₅, R₆, R₇, R₉, and R₁₀ are H and R₃ is (C₁-C₆)-alkoxyl, and R′ is H.

17. The compound according to claim 3, wherein R₃ is (C₁-C₃)-alkyl.

18. A composition comprising the compound as defined in claim 4, wherein the composition is either
(i) a composition selected from the group consisting of a cosmetic composition, a pharmaceutical composition, and a personal care composition and comprises at least an effective amount of the compound as defined in claim 4 for the protection of either a human or animal living body from ultraviolet radiation, together with one or more pharmaceutically or cosmetically acceptable excipients or carriers; or alternatively,
(ii) a composition comprising at least an effective amount of the compound as defined in claim 4 for the protection of a material from ultraviolet radiation, together with appropriate components selected from at least a polymer, a solvent an additive, and mixtures thereof.

19. A composition comprising the compound as defined in claim 5, wherein the composition is either
(i) a composition selected from the group consisting of a cosmetic composition, a pharmaceutical composition, and a personal care composition and comprises at least an effective amount of the compound as defined in claim 5 for the protection of either a human or animal living body from ultraviolet radiation, together with one or more pharmaceutically or cosmetically acceptable excipients or carriers; or alternatively,
(ii) a composition comprising at least an effective amount of the compound as defined in claim 5 for the protection of a material from ultraviolet radiation, together with appropriate components selected from at least a polymer, a solvent an additive, and mixtures thereof.
